(19) United States
(12) Patent Application Publication
(10) Pub. No.: US 2010/0310630 A1
(43) Pub. Date: Dec. 9, 2010

(54) COATED SURFACE FOR CELL CULTURE

(75) Inventor: Stephan Reichel, Gifhorn (DE)

Correspondence Address: GREER, BURNS & CRAIN, 300 S WACKER DR, 25TH FLOOR, CHICAGO, IL, 60606, US

(73) Assignee: TECHNISCHE UNIVERSITAT BRAUNSCHWEIG, Braunschweig (DE)

(21) Appl. No.: 12/597,641

(22) PCT Filed: Apr. 27, 2003
(86) PCT No.: PCT/EP2007/054198

§ 371 Date: Jul. 7, 2010

(51) Int. Cl. A61K 9/00; B05D 3/02; C12N 5/071; A61K 35/12; A61P 17/02; A61P 27/02

(52) U.S. Cl. 424/427; 427/384; 435/395; 435/371; 435/366; 424/93.1

(57) ABSTRACT

The invention makes available a keratin coating of a support substrate and a method for the production thereof. This keratin coating is suitable particularly for the in vitro or in vivo culturing of epithelial or endothelial cells and, because of its optical transparency, for microscopy. The keratin coating is prepared by applying or coating keratin in the form of nanoparticles from an aqueous keratin solution or keratin suspension. The solution or suspension does not contain any reducing compounds. The keratin solution is prepared preferably from hair, such as human hair. For the preparation of the coating, keratin is brought into solution or into a suspension of nanoparticles by mixing the keratin with an aqueous composition which contains thiourea, urea, and mercaptoethanol.

The present invention relates to a coated surface of a natural or synthetic substrate, the method for producing the coated surface, and the use of the coated surface for promoting cell growth in vitro or in vivo. The support substrate can be a natural or synthetic polymer, for example a plastic such as polystyrene, polyethylene, polypropylene, polybutylene, polyacrylate, polycarbonate, and copolymers of these, or can be a mineral support substrate, for example ceramic or glass.

When the surface coated according to the present invention is used for in vitro culturing of animal cells, the surface is used as a part of the culture vessel; for use in vivo, the surface coated according to the present invention is used in the production of medical products in order to promote wound healing, for example in the form of wound dressings. Surfaces coated according to the present invention, or forms made of the material of the coating according to the present invention, can also be used to produce implants that include cells cultured on the surface and that are used for example as a replacement for tissue or tissue layers, such as the cornea.

### BACKGROUND OF THE INVENTION

DE 698 08 291 T2 discloses a wound dressing having a surface that has a cell anchoring layer that can be biodegraded, made of heparin, inositol phosphate, fucoidin, syndecan, betaglycan, perlecan, dextran sulfate, pentosan, mesoglycan, polyvinyl sulfate, or polylysine.

AT 412 781 B describes molded bodies made of plastic filled with biological fibrous material, e.g. with starch, corn or rice meal, gluten, collagen, keratin, lignin, pectin, and hemicelluloses. The processing takes place through injection molding.

WO 95/01998 describes thermoplasts for producing cell culture vessels to which a thermostable polypeptide is added, for example pronectin,.

Parmar et al. (American Journal of Ophthalmology 299 ff, (2006)) describe the in vitro culturing of human amniotic epithelial cells on the concave side of a collagen form in order to produce a transplant as a replacement for the cornea.

Talbot (Molecular Vision, 65-75 (2006)) describes the production of corneal epithelial cells through the culturing of rabbit limbic epithelial cells on a fibrin gel matrix.

For cell culturing, vessels made of polymers are known that have a coating made of laminin, fibronectin, collagen, or polylysine. Thus, Yamauchi et al. (J. of Biomedical Mat. Res. 31, 439-444 (1996)) describe the coating of vessels for cell culturing with a keratin solution that was produced through degreasing of sheep wool, incubation in concentrated urea solution with SDS and 2-mercaptoethanol at neutral pH for 12 hours at 50° C. and subsequent dialysis against 0.08% by weight 2-mercaptoethanol in water. For the coating, 0.08 mL of 50% glycerin was added to 10 mL of the solution of reduced keratin, and the result was applied to 40 cm² and dried.

Yamauchi et al, (J. Biomater, Sci. Polymer Edn, 9: 259-270 (1998)) describe the culturing of L929 fibroblasts in polystyrene plates that were coated with a keratin solution of sheep wool in 7 M urea, 2-mercaptoethanol, and optionally SDS. Cell growth was observed only in the absence of SDS. The keratin film did not contain any glycerin. The data concerning the cell culturing relate only to the first 48 hours after seeding.

Tanabe et al. (Mat. Sc. and Engineering C 24 (2004) 441-446) describe a coating produced from reduced keratin solution and optionally 10-30% chitosan for cell culturing.

### OBJECT OF THE INVENTION

Against the background of the known prior art, the object of the present invention is to provide a coating for substrate surfaces that brings about improved cell growth and/or permits a better optical monitoring of cells cultured on the coating. For use in in vitro culturing of animal cells, the seeding efficiency and/or reproduction rate of the cells and/or the saturation density that is to be achieved are to be increased thereby. For use for producing pharmaceutical products, for example wound dressings, or for use for producing implants, an improvement of tissue regeneration is sought.

### GENERAL DESCRIPTION OF THE INVENTION

The present invention achieves the above-named objects by providing a coated surface of a support substrate and a method for producing a coated support substrate, the coating of the support substrate having keratin, preferably essentially consisting of keratin. The advantageous properties of the keratin coating according to the present invention are seen in particular in use for the in vitro or in vivo culturing of epithelial or endothelial cells.

The keratin coating is produced by applying or coating with keratin, preferably in the form of nanoparticles from an aqueous keratin solution or keratin suspension, the solution or suspension containing no reducing compounds and having preferably been dialyzed against water containing no additives. Here, the keratin used to produce the solution is preferably α-keratin. Particularly preferably, the keratin solution is produced from hair, for example human hair. In order to produce the coating, keratin is brought into solution, or into nanoparticulate suspension, by mixing with an aqueous composition, the aqueous composition preferably containing only thiourea, urea, and mercaptoethanol. In this aqueous composition, mercaptoethanol may be replaced by some other reducing compound.

By applying keratin from the aqueous keratin solution or suspension to the substrate surface, an optically transparent keratin layer can be produced, so that given an optically transparent support substrate, for example glass or polystyrene, an optically transparent culture vessel for the cell culture can be produced that permits a significantly more effective cell culturing due to the keratin coating.

The analysis of the size distribution of the solution or nanoparticulate suspension of keratin used according to the present invention to produce the keratin coating, also designated aqueous composition of keratin or solubilized keratin, shows that the method according to the present invention for the dissolving or solubilizing produces a very narrow, monomodal size distribution around approximately 50-250 nm. Using photon correlation spectroscopy by means of dynamic laser light scattering, a polydispersity index can be analyzed that is significantly smaller than that for keratin solutions known from the prior art. Correspondingly, keratin layers produced from the keratin solution are preferred that have a monomodal size distribution whose main value is in the range from 20 to 5000 nm, preferably 100 to 1000 nm, more preferably 100 to 200 nm.

In comparing the keratin coating according to the present invention with other peptide coatings in vessels for cell culture, it has turned out that at least for some cell types of immortalized cell tines and of primary cells, a higher seeding efficiency and/or better growth was obtained. At present, it is assumed that the significantly improved culturing results are due to the particular structure of the keratin coating, which has a nanoparticulate structure.

In use for the manufacture of pharmaceutical products, for example a corneal implant, the keratin coating according to the present invention enables a support substrate to be colonized with animal cells, for example human amniotic epithelial cells or corneal cells, such that the keratin coating could be detached from the support substrate after the colonization and is useable as an implant. Such an implant or transplant colonized with animal, in particular human, cells then does not have any support substrate other than the keratin, which provides the stability of the implant.

For the use of the keratin coating for manufacturing pharmaceutical products for medical purposes, e.g. as a transplant or implant, in a specific embodiment it is provided that a keratin film is used instead of the keratin coating on a support substrate. The keratin film without additional support substrate can be manufactured by producing the keratin film on a substrate and subsequently separating the keratin film from the substrate, for example by detaching a dried keratin film. Such a keratin film, in this specific embodiment, can, like the keratin coating, be produced from an aqueous solution w a suspension of keratin, and has the inventive properties of promotion of cell growth and/or high seeding efficiency. Such a keratin film is suitable in particular for use in the manufacture of implants that may also have animal cells adhering on one or two sides, applied onto the surface of the keratin film through in vitro culturing.

For use in the manufacture of wound dressings, a keratin film according to the present invention is preferably produced on a support substrate that determines the mechanical stability of the material bond. Possible support substrates include in particular polymers that are insoluble in water, for example from the group comprising polymer films, in particular films of polyethylene terephthalate (PET).

In the manufacture of wound dressings, it is preferable for the keratin film to contain compounds that promote cell growth, such as PDGF (platelet-derived growth factor), rhPDGF-BB (becaplermin), EGF (epidermal growth (actor), PDECGF (platelet-derived endothelial cell growth factor), aFGF (acidic fibroblast growth factor), bFGF (basic fibroblast growth factor), TGFβ (transformation growth actor β), TGFα (transformation growth factor α), KGF (keratinocyte growth factor), IGF1/IGF2 (insulin-like growth factors), TNF (tumor necrosis factor), and/or additives that improve the adhesion of tissue, such as laminin, fibronectin, and/or antibiotic ingredients such as antibiotics, iodine, and/or substances that promote wound healing, such as dexpanthenol.

### DETAILED DESCRIPTION OF THE INVENTION

The coating according to the present invention of keratin on a support substrate, or the keratin film without adhering support substrate, respectively, is preferably manufactured by producing a keratin film on a support substrate from an aqueous solution or suspension of keratin, with subsequent separation of the keratin film if necessary. The application of the keratin takes place by wetting a substrate surface with the aqueous keratin solution or suspension. The obtained coating of keratin on the surface of the support substrate contacted by the keratin solution is well-suited for culturing animal cells due to the high cell growth and the high seeding efficiency. Surprisingly, the optical transparency of the applied keratin layer does not significantly decrease as the layer thickness increases. Thin keratin films may have a high degree of transparency, and also retain the advantageous properties for cell culturing and wound heating of larger layer thicknesses.

Dependent on the support substrate, applied keratin adheres to the surface thereof, so that for example tor plastics or glass, used to manufacture vessels for cell culturing, a sufficiently stable adhesion of the keratin film is obtained without further additives. In a preferred specific embodiment, the surface of the support substrate that is to be coated can be coated with keratin through contacting with the aqueous keratin solution under conditions in which keratin precipitates from the aqueous solution or suspension. Such conditions include for example the presence of oxygen from air in contact with the aqueous keratin solution or suspension, the keratin solution having no reducing components.

Dependent on the concentration of keratin in the aqueous solution, a keratin coating according to the present invention can be produced on the surface of the support substrate through the contacting of the support substrate with aqueous keratin solution or suspension for a time period in the range from 5 seconds to 10 minutes of simple, or, with interspersed periods of drying, multiple contacting. Here it has turned out that for the optical transparency it is advantageous to produce a keratin coating that is as thin a layer as possible, while for the specific embodiment in which a keratin film is produced and used it is advantageous for the mechanical stability to produce a larger layer thickness through simple, preferably multiple application of the aqueous keratin solution or suspension onto a surface. In the specific embodiment of the present invention as a keratin film without an adhering support substrate, the deposited keratin can be removed from the support substrate after hardening of the deposited keratin. The hardening of the keratin film is achieved by drying off the solution water.

Preferred layer thicknesses of the keratin film according to the present invention on a support substrate are in the range from 0.1 to 1 μm, preferably 0.2 to 0.6 μm; for keratin films, in the range from 1 to 100 μm, preferably 1 to 50 μm, more preferably 2 to 20 μm.

In order to manufacture the keratin solution that contains, in addition to dissolved keratin, suspended nanoparticles of keratin, and which for the purposes of the present invention is equivalently referred to as a keratin suspension, from which the keratin coating according to the present invention and the keratin film can be produced, a-keratin of natural origin, preferably from human hair, is preferably used. The α-keratin is brought into solution in water for example through urea and mercaptoethanol, preferably in combination with thiourea. Undissolved components can be removed by centrifuging at 10,000×g, and optionally through alternative or additional filtration.

Urea, mercaptoethanol, and/or thiourea are separated essentially from the keratin-containing fraction through extensive dialysis against distilled water. The dialysate has a size distribution Z_{mean 50%} of 109 nm; in some examples, D₁₀<84 nm and D₉₀>140 nm were found.

This keratin solution, which in the present application is also designated as a suspension of nanoparticles of keratin, is contacted with the surface that is to be coated of a support substrate, excess material is removed, and the wetted surface is allowed to dry. This method of contacting and drying can be repeated in order to produce a thicker keratin layer. The thickness of the obtained keratin film increases both with the keratin concentration of the solution used and also with the increase in the volume of the keratin solution from which solution water on the support substrate is removed.

The obtained keratin film is transparent to visible light. In electron microscopy, nanostructures are visible, which in the present application are also designated nanoparticles. The deposited keratin film contains essentially no free thiol groups, and it is assumed that these are already essentially completely oxidized in the dialysate, i.e. after removal of the reducing compounds added to the keratin.

In the culturing of cells in vitro in cell culture vessels whose surfaces were coated with a keratin film according to the present invention, in addition to a higher seeding efficiency a faster doubling rate and a significantly higher saturation density was determined for a large number of cell types.

The cells cultured on a keratin-coated support substrate are suitable for in vitro trials for the permeation of active substances through a cell layer, e.g. if the support substrate is a polycarbonate filter having pores in the range from 0.4 to 3 μm, i.e. is itself diffusion-permeable. The cells were cultured on one side of the polycarbonate filter coated with keratin.

In a further preferred specific embodiment, the keratin film is cross-linked, for example by contacting the keratin nanoparticles deposited onto a support substrate from the keratin suspension with a cross-linking agent, e.g. reagent, having at least two functional groups that are reactive with keratin. Suitable cross-linking reagents have for example at least two carbonyl groups and/or imide groups. It has turned out that glutaraldehyde and carbodiimides, e.g. 1-ethyl-3-(3-dimethytaminopropyl)carbodiimide, or succinimides, e.g. N-hydroxysuccinimide, are suitable. Following the contacting, non-converted cross-linking reagent is removed or, preferably, by increasing the temperature to up to 200° C. is removed or converted to products that are harmless to the cell culture.

The cross-linking results in an increase in the mechanical stability of the keratin film, either as a coating of a support substrate or, after detachment from the support substrate, as a single-layer keratin film.

Alternatively or in addition to the contacting of the keratin film deposited from the suspension with cross-linking reagent, an increase in the mechanical stability of the keratin film can be achieved by increasing the temperature, e.g. during or after removal of the solution water, to up to 200° C., preferably to 80 to 180° C., more preferably to 100 to 130° C., for a time period from 1 to 30 minutes, preferably 2 to 15 minutes.

In the following, the present invention is explained in more detail on the basis of examples with reference to the Figures, in which

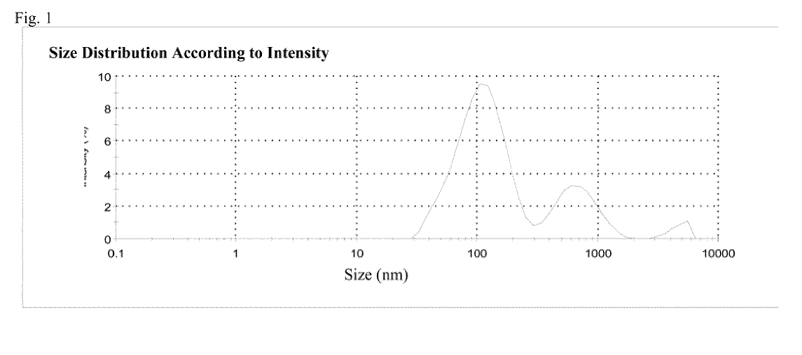

FIG. 1 shows the measurement result of the photon correlation spectroscopy of the keratin solution according to comparative example 2 (y-axis shows intensity in %),

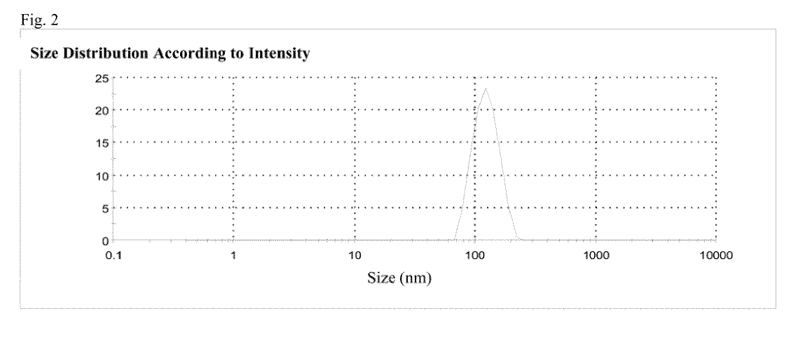

FIG. 2 shows the measurement result of the photon correlation spectroscopy of the keratin solution according to example 1 (y-axis shows intensity in %),

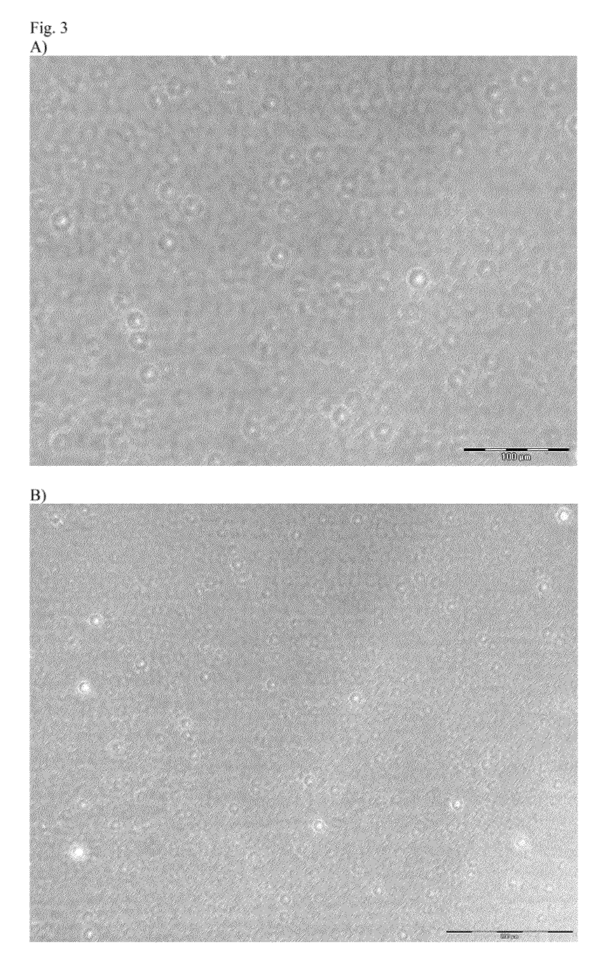

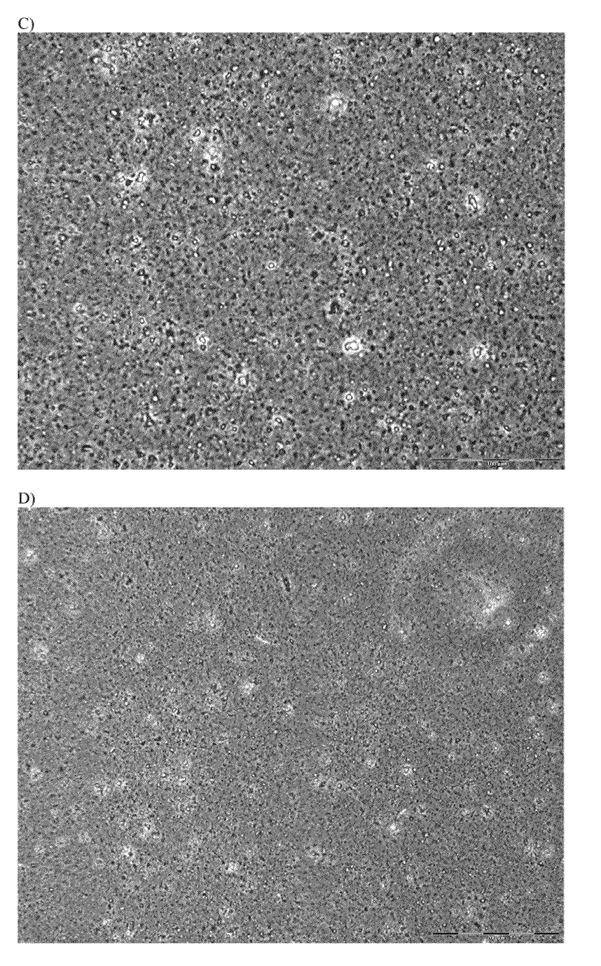

FIG. 3 shows light microscope pictures of: A) a keratin layer according to the present invention; scale bar=100 μm; B) a keratin layer according to the present invention, bar=200 μm, and a keratin layer according to comparative trial 2 with: C) enlargement for bar=100 μm, D) enlargement for bar=200 μm, E) enlargement for bar=500 μm;

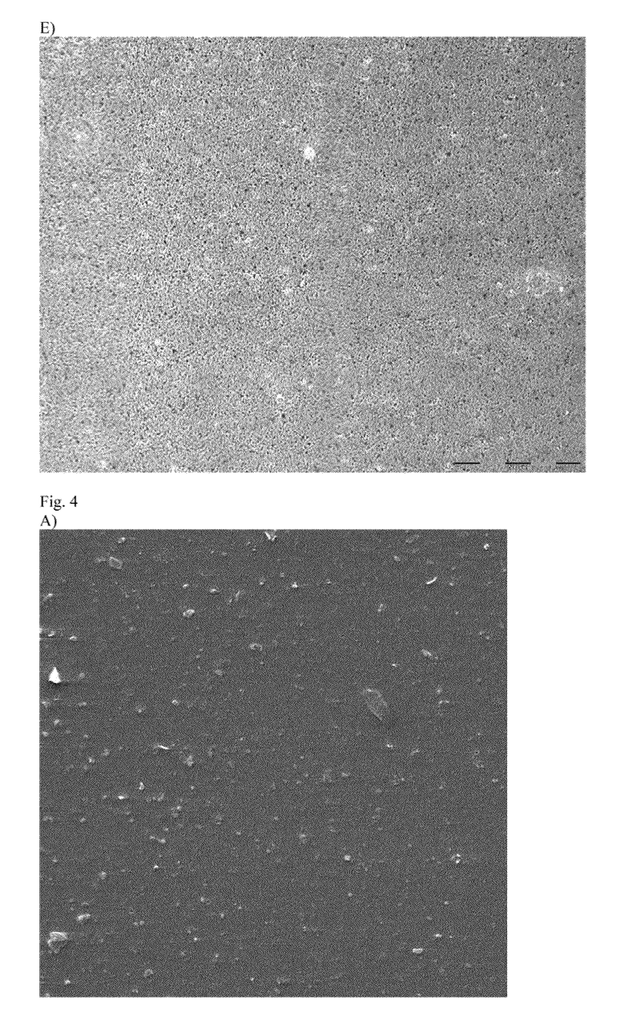

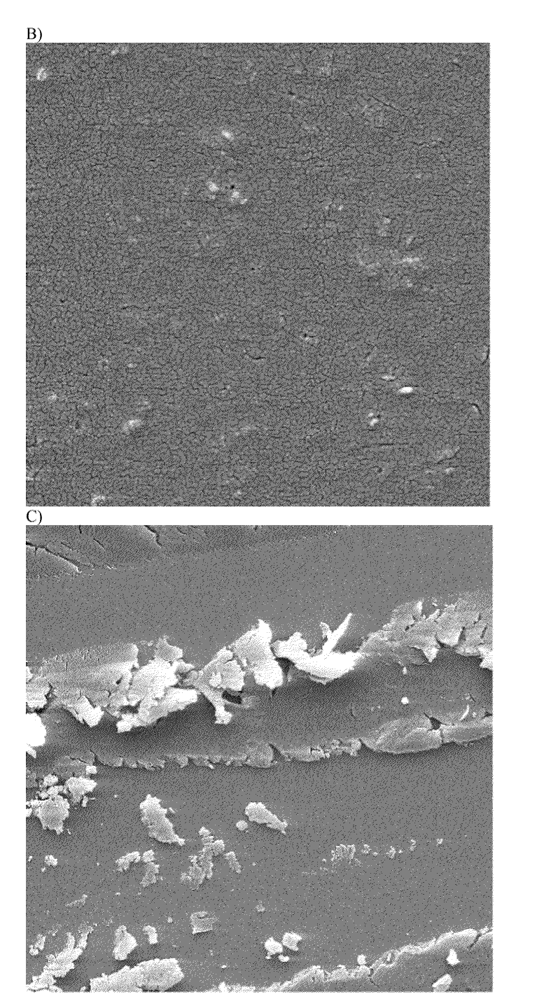

FIG. 4 shows scanning electron microscope pictures of keratin layers according to comparative trial 2, namely with A) 90 μm edge length, B) 18 μm edge length, C) after scoring, 90 μm edge length, and D), as an excerpt of C), 30 μm edge length.

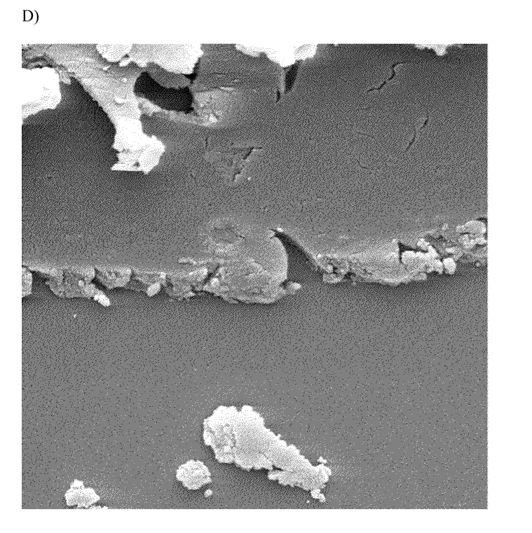

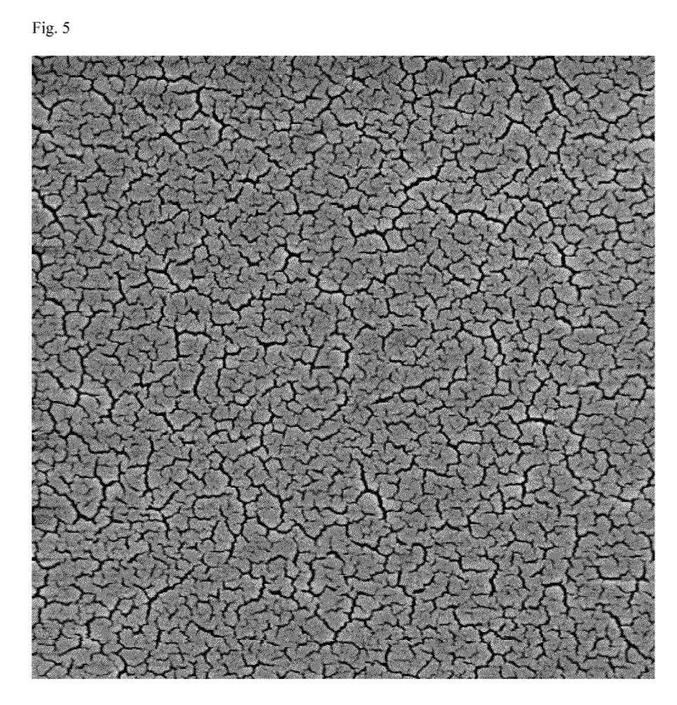

FIG. 5 shows a scanning electron microscopic picture of a keratin film according to the present invention,

FIG. 6 shows a scanning electron microscopic picture of a keratin film according to the present invention after detachment in some areas from the support substrate,

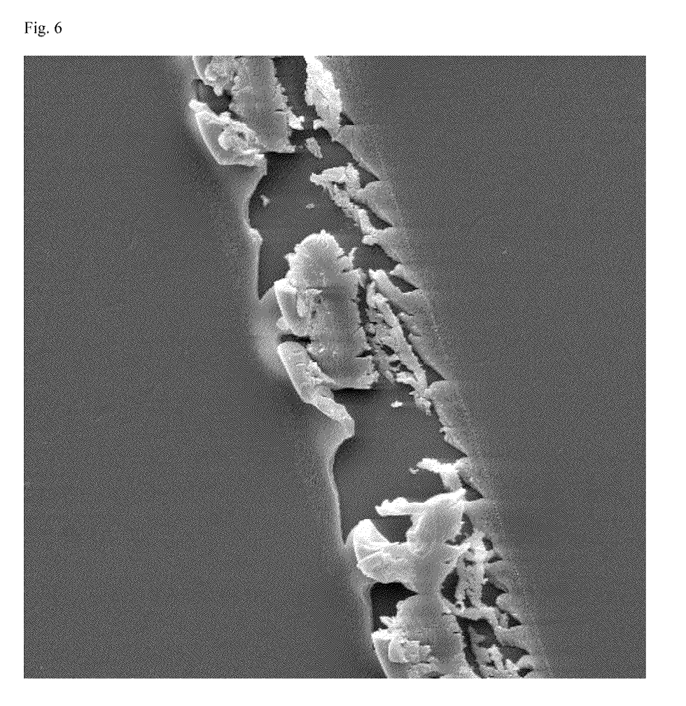

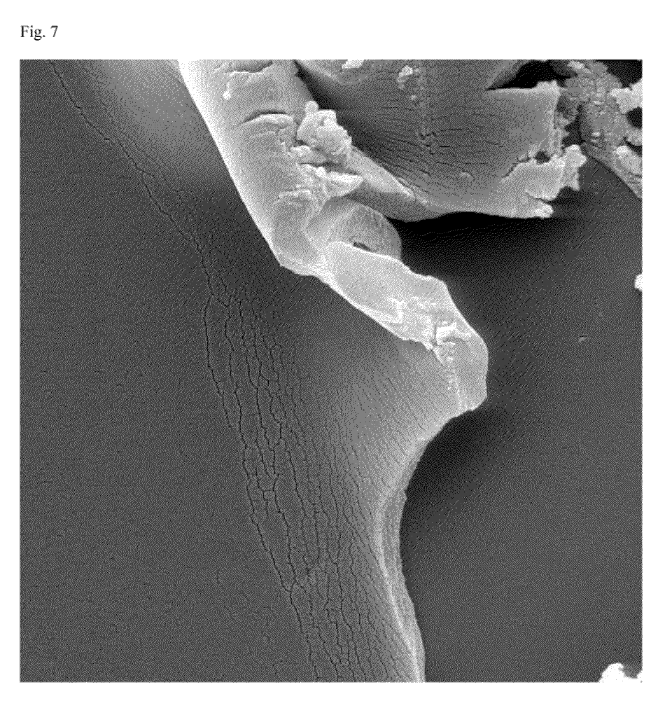

FIG. 7 shows a scanning electron microscope picture of an excerpt from FIG. 6,

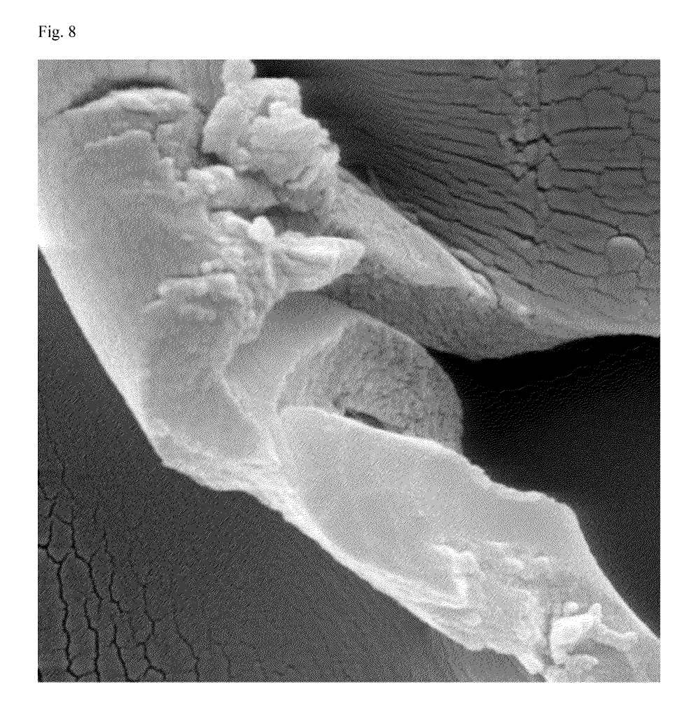

FIG. 8 shows an excerpt from FIG. 7, and

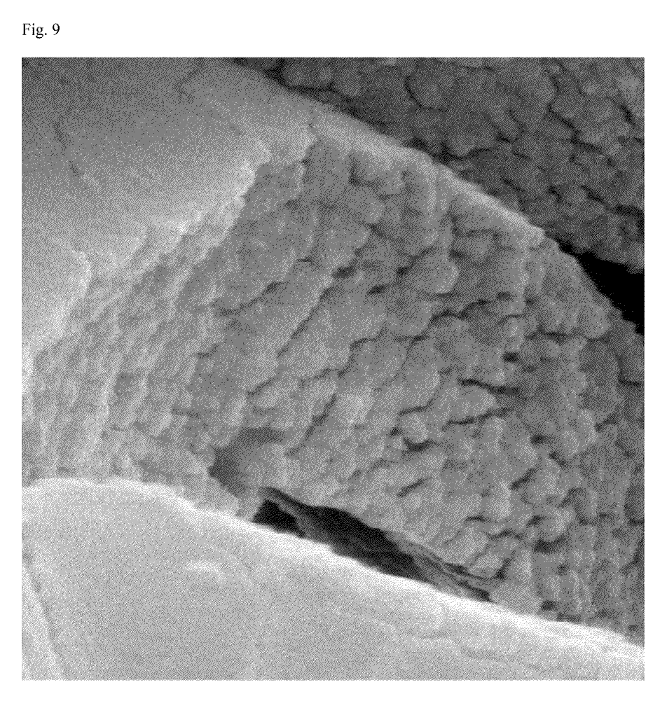

FIG. 9 shows an excerpt from FIG. 8.

### COMPARATIVE EXAMPLE 1

### Manufacture of a Plastic Surface with a Keratin Coating Through Precipitation with Trichloroacetic Acid (TCA)

For an aqueous keratin solution, 20 g of human hair was washed with a 0.5% SDS solution, dried, and degreased through incubation with n-hexane overnight. After removal of the hexane, 400 mL of a 25 mM Tris solution (pH 8.5) with 2 M thiourea; 5 M urea, and 5% mercaptoethanol in water was added. After sealing the vessel with Parafilm, agitation took place for 72 hours at 50° C. Undissolved components were removed by centrifuging in a laboratory centrifuge at approximately 10,000×g (10 minutes, 5000 RPM); the supernatant was additionally filtered using a filter having a pore size of 2.5 μm.

The keratin solution is pipetted into the wells of a microtiter plate (polystyrene), in which a 5% by weight solution of TCA in water is already present. This results in a white precipitation of the protein. The precipitate is given time to settle, and the supernatant is then removed and the plate is dried. A white, optically opaque film remains. In order to remove the TCA, the dried film is washed multiple times with distilled water. In comparative trials with uncoated microtiter plates, no significant improvement, or only a slight improvement, was observed in the growth rate or seeding efficiency in the animal cell culture, while the microtiter plates (according to Example 1) according to the present invention showed improved values for growth rate and seeding efficiency for a large number of cell lines. The measured values are shown in the following Table 1 of Example 2.

### COMPARATIVE EXAMPLE 2

### Manufacture of a Plastic Surface with a Keratin Coating from a Keratin Solution in the Presence of Reducing Compounds

A plate coated with reduced keratin for cell culture was produced according to Yamauchi et al. (J. Biomater. Sci. Polym. Ed 9: 259-270 (1998)) without using SDS in the keratin solution.

Specifically, human hair (20 g) was agitated in 360 mL 7 M urea with 32 mL 2-mercaptoethanol, in a closed glass bottle at 50° C. for 24 hours, and was subsequently filtered. The filtrate was dialyzed overnight three times against 12 L distilled water having 0.2% by weight 2-mercaptoethanol. A cloudy solution of approximately 480 mL was obtained. The analysis of the particle size distribution, using photon correlation spectroscopy, is shown in FIG. 1, and resulted in a multimodal size distribution having a Z-average value about a particle size of approximately 125 nm, and, due to a strongly multimodal distribution, a polydispersity index of 0.41. The distribution curve shows three peak values, at approximately 115 nm, 700 nm, and 4770 nm.

The application of this keratin solution according to Yamauchi took place according to Example 1.

Light microscope pictures are shown in FIG. 3 alongside pictures of the plates coated according to the present invention. The pictures of FIG. 3, which, as C), D), and E), show the polystyrene surfaces coated according to this comparison, clearly are provided with irregularities that present a non-homogenous background for microscopic purposes. The keratin films according to the present invention are shown as FIG. 3, A) and B), and are significantly more homogenous under the same microscopy conditions.

Electron microscope pictures are shown in FIG. 4, A)-D), in which uneven areas of this keratin coating are conspicuous, while the keratin film according to the present invention, of which electron microscope pictures are shown in FIGS. 5-9, is significantly more homogenous, with a flat surface.

### EXAMPLE 1

### Manufacture of a Plastic Surface Having a Keratin Coating

In order to manufacture a keratin coating on a support substrate, and a keratin film that is detachable from a support substrate, respectively, keratin is deposited from an aqueous keratine solution. The aqueous solution was degreased through washing of 20 g of human hair with a 0.5% SDS solution, drying, and was degreased through incubation with n-hexane overnight. After removal of the hexane, 400 mL of a 25 mM Tris solution (pH 8.5) with 2 M thiourea, 5 M urea, and 5% mercaptoethanol in water is added. After seating the vessel with Parafilm, agitation takes place for 72 hours at 50° C. Undissolved components are removed by centrifuging in a laboratory centrifuge at approximately 10,000×g (10 min, 5000 RPM); the supernatant was additionally filtered using a filter having a pore size of 2.5 μm. The filtrate was capable of being stored at 4° C., or frozen in aliquots, without essential changes in its properties.

The filtrate was dialyzed against distilled water, e.g. using a Spectrapore 1 membrane (exclusion limit 6-8000 Da), standardly dialyzing each 100 mL filtrate against 5 L water over 72 h, while changing the water 6 times. The dialysate was centrifuged in an ultracentrifuge for 10 min at 15,000 RPM in order to remove aggregates. The centrifugate can be used immediately to produce coated support substrates or in the production of keratin films.

The measurement result of the photon correlation spectroscopy for particle size distribution is shown in FIG. 2, and shows a narrow monomodal size distribution having a Z-average (Z mean value) of 120 nm and a polydispersity index of 0.07.

The protein concentration of the keratin solution according to the present invention, measured according to Bradford, was approximately twice as high as that of comparative example 2 according to Yamauchi.

As an example of a support substrate, microtiter cell culture plates made of polystyrene or polycarbonate were used, and were contacted with a volume sufficient to continuously wet the surface. For 24-well plates for cell culturing, 400 μL coating solution was pipetted into each well. Immediately after this pipetting, i.e. after about 5 to 10 s, the solution was completely removed and the surface was dried in air under sterile conditions. This contacting and subsequent drying was repeated 2-5 times. After the drying, the plates are stable and can be stored at room temperature.

A sterilization of the coated surfaces can take place by irradiation or by wetting in 70% ethanol/water for two hours with subsequent drying.

Light microscopic views of a keratin coating manufactured in this way are shown in FIGS. 3A) and B), in which the enlargement is indicated by the scale of the burned-in dimension bar. In comparison with Figures C)-E), which show keratin coatings according to the comparative example, the significantly increased homogeneity and optical transparency of the keratin film according to the present invention are clearly seen. FIG. 5 shows a scanning electron microscope picture of a keratin film according to the present invention in wells of a microtiter plate (polystyrene) having a picture edge length of approximately 18 μm.

FIG. 6 shows a segment of a coated surface in which the applied keratin coating has been detached in sonic sections by scratching with a needle. The edge length of the electron microscope picture in FIG. 6 is approximately 180 μm. FIG. 7 shows a segment of the picture of FIG. 6, approximately in the center thereof, having a picture edge length of approximately 45 μm.

Another enlargement of part of FIG. 7, approximately in the center of the upper third, is shown in FIG. 8, in which the picture edge length is approximately ˜μm; another enlargement of a part of FIG. 8, approximately in the center of the picture, is shown in FIG. 9 (picture edge length 4.5 μm).

The Figures show that the keratin layer produced according to the present invention contains nanoparticles or substructures having diameters of approximately 0.3 to 0.4 μm. If the aqueous keratin solution is applied four to five times, with drying between applications, keratin layer thicknesses of approximately 1.5 to 4 μm are produced.

Structural differences from keratin coatings of reduced keratin according to Yamauchi et at, which were produced in comparative example 2, also include, in addition to the monomodal particle size distribution, the improved optical transparency and the significantly lower number of disturbing irregularities, resulting from the method of producing the keratin film according to the present invention.

### EXAMPLE 2

### Culturing of Animal Cells on Plastic Surfaces with Keratin Coating

Culture vessels for cell culture produced according to Example 1, made of polystyrene with a keratin coating, were each seeded with approximately 30,000 cells alter trypsinization in fresh cell culture medium. In order to obtain counts, the cells were detached from three wells each at simultaneous points in time, by trypsinization, and were counted in a Coulter counter. From the obtained values, a growth curve was produced (logarithm of the cell count over growth time). From the growth curve, sigmoidal fitting was used to graphically determine the lag phase, the population doubting time (PDT), and the achieved saturation density.

In order to determine the seeding efficiency, the wells were each seeded with 100,000 cells per well in cell culture medium. After culturing over a period of 14 hours, the medium was suctioned out, the well was flushed, and the attached cells were detached by trypsinization and were counted. The seeding efficiency results as the quotient of the number of attached cells divided by the number of cells originally used.

The results are shown in the following tables. Table 1 shows the seeding efficiency in percent, and Table 2 shows the proliferation properties, For comparison, in each case uncoated identical culture vessels made of polystyrene were used (“polystyrene”).

TABLE 1
Comparison of seeding efficiency on uncoated polystyrene microtiter
plates with polystyrene microtiter plates keratin-coated according
to the present invention and with polystyrene microtiter plates keratin-
coated by TCA precipitation; indications in % of seeded cells

| | | | Keratin coating | | Keratin coating | |
|---|---|---|---|---|---|---|
| | Uncoated | | according to the | | with TCA | |
| | polystyrene | | present invention | | precipitation | |
| Cell line | MV | SD | MV | SD | MV | SD |
| Caco-2 | 16.1 | 3.3 | 42.3 | 8.6 | 26.1 | 3.1 |
| HaCaT | 49.7 | 6.1 | 75.3 | 8.5 | 48.5 | 8.2 |
| Sirc | 51.6 | 5.3 | 81.8 | 7.8 | 47.1 | 5.5 |
| Cepi | 29.7 | 2.4 | 81.4 | 10.6 | 32.7 | 2.9 |
| Cepi, serum- | 37.5 | 3.9 | 102.9 | 11.3 | 30.1 | 4 |
| reduced | | | | | | |
| Henc | 53.8 | 3.1 | 96.9 | 8.8 | 51.8 | 5.9 |
| HCK | 39.8 | 6.7 | 76.7 | 13.0 | 35.2 | 3.3 |
| Hufib | 36.9 | 4.4 | 101.2 | 13.4 | 33.3 | 1.8 |
| SZ 95 | 46.6 | 7.8 | 55.6 | 3.3 | 48.8 | 5 |
| HCE-T | 69.9 | 8.9 | 99.8 | 10.1 | 70.3 | 7.9 |
| PHK | 20.5 | 1.8 | 21.6 | 2.5 | 19.8 | 1.8 |

TABLE 2
Proliferation of cells on keratin-coated
surfaces compared to uncoated surfaces

| Cell line/parameter | Polystyrene | Keratin, clear* | TCA-precipitated** |
|---|---|---|---|
| Caco-2 | | | |
| Lag phase [d] | 1.3 | 0.95 | 3.5 |
| PDT [d] | 1.55 | 1.35 | 1 |
| Saturation density | 260,000 | 490,000 | 285,000 |
| [cells/cm²] | | | |
| HaCaT | | | |
| Lag phase [d] | 2.9 | 2.2 | 5.5 |
| PDT [d] | 1.3 | 1 | 0.7 |
| Saturation density | 200,000 | 435,000 | 140,000 |
| [cells/cm²] | | | |
| Sirc | | | |
| Lag phase [d] | 1.4 | 2.3 | 1.4 |
| PDT [d] | 1 | 1.2 | 1.1 |
| Saturation density | 530,000 | 1,180,000 | 430,000 |
| [cells/cm²] | | | |
| Cepi | | | |
| Lag phase [d] | 2 | 1.25 | 1.4 |
| PDT [d] | 0.9 | 1 | 0.9 |
| Saturation density | 290,000 | 970,000 | 300,000 |
| [cells/cm²] | | | |
| Cepi | | | |
| (serum-reduced) | | | |
| Lag phase [d] | 1.3 | 1.35 | 2.0 |
| PDT [d] | 1.2 | 1 | 1.3 |
| Saturation density | 160,000 | 425,000 | 200,000 |
| [cells/cm²] | | | |
| Henc | | | |
| Lag phase [d] | 2.7 | 2.6 | 2.7 |
| PDT [d] | 1.3 | 1.3 | 1.8 |
| Saturation density | 320,000 | 605,000 | 100,000 |
| [cells/cm²] | | | |
| HCK | | | |
| Lag phase [d] | 0.9 | 0.95 | 0.9 |
| PDT [d] | 0.85 | 0.85 | 2.0 |
| Saturation density | 540,000 | 805,000 | 500,000 |
| [cells/cm²] | | | |
| Hufib | | | |
| Lag phase [d] | 1.6 | 1.9 | 2.3 |
| PDT [d] | 1.7 | 1.5 | 2.0 |
| Saturation density | 75,000 | 180,000 | 45,000 |
| [cells/cm²] | | | |
| SZ 95 | | | |
| Lag phase [d] | 2.6 | 3.4 | 4.0 |
| PDT [d] | 0.9 | 1.4 | 1.2 |
| Saturation density | 160,000 | 64,000 | 100,000 |
| [cells/cm²] | | | |
| HCE-T | | | |
| Lag phase [d] | 1.2 | 0.9 | 1.1 |
| PDT [d] | 0.9 | 0.8 | 1.7 |
| Saturation density | 156,000 | 960,000 | 300,000 |
| [cells/cm²] | | | |
| PHK | | | |
| Lag phase [d] | 1 | 2.7 | 2.0 |
| PDT [d] | 0.9 | 0.9 | 1.3 |
| Saturation density | 105,000 | 105,000 | 100,000 |
| [cells/cm²] | | | |

*keratin clear = keratin coating according to the present invention on polystyrene
**TCA-precipitated = keratin coating according to comparison example 1 on polystyrene

2: Caco-2 cell line (colon carcinoma, human), immortalized

HaCaT: HaCaT cell line (epidermis, human), immortalized

Sirc: SIRC cell tine (corneal epithelium, rabbit), immortalized

Cepi: Cepi cell line (corneal epithelium, human), immortalized

Cepi (serum-reduced): as above, cultured with lower serum content

Henc: HENC cell line (corneal endothelium, human), immortalized

HCK: HCK corneal fibroblasts, human), immortalized

Hufib: HUFIB (corneal fibroblasts, human), primary cultures

SZ 95: SZ 95: (sebocytes, human), immortalized

HCE-T: HCE-T (corneal epithelium, human), immortalized

PHK: PHK (keratinocytes, human), primary culture

Both from the values for the seeding efficiency and from the values for the proliferation behavior, i.e. the shortening of the lag phase and the shortening of the population doubling time, and the significant increase in the saturation density of the tested cells, it is clear that culturing using the keratin coating according to the present invention is significantly improved compared to uncoated microtiter plates and compared to plates coated with keratin by TCA precipitation.

In contrast to the keratin coating according to the present invention, the culturing of epithelial and endothelial cells on keratin-coated plates produced from keratin according to comparative example 2 showed significantly worse values for the proliferation behavior, the shortening of the population doubling time, and the saturation density. The values for seeding efficiency and proliferation were determined using the cell line HCE-T:

TABLE 3
Comparison of the properties of keratin films for cell culture

| | Keratin film | | | | | |
|---|---|---|---|---|---|---|
| | according to | | | | | |
| | comparative | | | | | |
| | trial 2 | | Keratin clear* | | Polystyrene | |
| Lag phase [d] | 1.0 | | 0.9 | | 1.2 | |
| PDT [d] | 0.85 | | 0.8 | | 0.9 | |
| Saturation | 820,000 | | 960,000 | | 156,000 | |
| density | | | | | | |
| [cells/cm²] | | | | | | |
| Seeding | MV = | SD = | MV = | SD = | MV = | SD = |
| efficiency [%] | 83.3 | 7.2 | 99.8 | 10.1 | 69.9 | 8.9 |

It is presumed that a reason for the more homogenous surface and the better proliferation behavior of cultured animal cells lies in the structure of the keratin film produced by the method according to the present invention, in particular in the solubilization of hair in the presence of thiourea and the absence of 2-mercaptoethanol during the dialysis, while according to comparative trial 2 the solubilization took place without thiourea and the dialysis took place against water containing 2-mercaptoethanol.

### EXAMPLE 3

### Culturing of Cells on Keratin-Coated Surfaces for Use in the Measurement of the Permeation of Pharmaceutical Compounds Through Cell Layers

For the in vitro measurement of the permeation of pharmaceutical compounds through cultured cells, cells were grown on polycarbonate filters coated with keratin according to the present invention, the polycarbonate filters themselves being diffusion-for useable due to pores having sizes in the range from 0.4-3 μm. According to Example 2, on the polycarbonate filter a keratin coating was produced on which cells were in turn were cultured in one layer or multiple layers under cell culture conditions.

The cells grown on the keratin layer of the polycarbonate filter were successfully used to measure the diffusion of pharmaceutical compounds through the cell layers. As an example of a pharmaceutical compound, Na-fluorescein was used, applied onto one side of the cultured cells. The permeation through the cell layers was determined using fluorescence spectroscopy.

In comparison to a trial that was identical except for the lack of a keratin coating, it was found that the keratin layer on the polycarbonate filter has no influence, or has a constant influence, on the permeation rates of the pharmaceutical compound, so that this influence of the keratin coating can be determined and computationally removed through comparison with a comparison trial with cultured cells on an uncoated polymer filter.

### EXAMPLE 4

### Production of a Wound Dressing

For production of a wound dressing having a keratin coating according to the present invention, as an example for an elastic support substrate a mixture having 60% by weight polyvinyl pyrrolidone, 35% by weight polyethylene glycol 400, and 5% by weight sodium carboxymethylcelluose was provided with a keratin layer corresponding to Example 1.

As a softener, glycerol in an aqueous solution was optionally applied and the water was removed by drying; alternatively, polyethylene glycol and/or polypropylene glycol were applied.

The support substrate exhibited the adhering keratin layer and was capable of being used to cover wounds.

### EXAMPLE 5

### Manufacture of a Keratin Film

A keratin film for use in the manufacture of pharmaceutical compositions, for example for producing wound dressings or for producing implants or transplants, was produced by depositing a keratin film according to Example 1 on a support substrate. Here, as a support substrate a polymer was preferably used having only slight adhesion to the keratin film deposited thereon, such as siliconized PET.

In order to increase the elasticity of the keratin layer, after the deposition the keratin layer was provided with a softener, or the softener was already added to the aqueous keratin solution. Suitable softeners include in particular glycerol, polyethylene glycol, polypropylene glycol, and mixtures thereof.

Following the drying of the keratin coating on the support substrate, preferably after the addition of softeners, the keratin film was obtained through mechanical removal from the support substrate.

Such a keratin film is suitable for use as a wound dressing or for the manufacture of an implant.

In order to increase the mechanical stability, following the deposition of the keratin film a cross-linking of the keratin particles took place through application of a 4% by weight glutaraldehyde solution, incubation at room temperature for 12 hours, subsequent removal of the glutaraldehyde, and washing with water. The resulting keratin film can be dried at room temperature or can be used in hydrated form.

Similar increases in the stability of the keratin film could be achieved in the absence of an added cross-linking reagent by drying at temperatures of 80 to 120° C. for 20-40 minutes. The drying at these temperatures could also be combined with contacting with cross-linking reagent.

### EXAMPLE 6

### Manufacture of an Implant with Animal Cells on a Keratin Film

As an example of an implant having a keratin film according to the present invention, rabbit corneal cells isolated according to the method of Talbot et al. (Molecular Vision 65-75 (2006)), or human amniotic epithelial cells isolated according to Parmar et al. (American J. of Ophthalmology, pp. 299-300 (February 2006)), were applied on one side of a segment of a keratin film manufactured according to Example 5 under cell culture conditions until confluence. Such a layer of corneal cells on a keratin film was then ready for use as a transplant for corneal replacement.

1. A method for manufacturing a keratin layer for the culturing of animal cells, the method comprising solubilizing hair in an aqueous composition of thiourea, urea, and 2-mercaptoethanol at pH 8-9, separation of thiourea, urea, and 2-mercaptoethanol from the aqueous composition of solubilized keratin, contacting of a support substrate with the aqueous composition, and subsequent drying.

2. The method for manufacturing a keratin layer as recited in claim 1, wherein the keratin has particles having an essentially monomodal size distribution in a range from 20 to 5000 nm.

3. The method for manufacturing a keratin layer as recited in claim 1, wherein the keratin is α-keratin.

4. The method for manufacturing a keratin layer as recited in claim 1, further comprising a step of separating the keratin layer from the support substrate.

5. The method for manufacturing a keratin layer as recited in claim 1, wherein the keratin layer contains PDGF (platelet-derived growth factor), rhPDGF-BB (becaplermin), EGF (epidermal growth factor), PDECGF (platelet-derived endothelial cell growth factor), aFGF (acidic fibroblast growth factor), bFGF (basic fibroblast growth factor), TGFβ (transformation growth factor β), TGFα (transformation growth factor α), KGF (keratinocyte growth factor), IGF1/IGF2 (insulin-like growth factors), TNF (tumor necrosis factor), and/or laminin, fibronectin, and/or antibiotic ingredients and/or substances that promote wound healing.

6. The method for manufacturing a keratin layer as recited in claim 1, wherein the keratin layer is cross-linked by a reagent containing at least two functional groups that are reactive with keratin.

7. The method for manufacturing a keratin layer as recited in claim 1, wherein the keratin layer is cross-linked by drying at a temperature from 80 to 200° C.

8. A method for manufacturing a keratin layer as recited in claim 1, wherein the hair comprises degreased hair and the aqueous solution comprises 2 M thiourea, 5 M urea, 5% by weight 2-mercaptoethanol at 50° C. and pH 8 to 9, the method further comprising dialysis against water containing no reducing compounds subsequent to said separation and prior to said contacting and drying.

9. The method as recited in claim 8, wherein the deposited keratin layer is contacted with a reagent having at least two functional groups that are reactive with keratin, and/or is dried at a temperature of 80 to 200° C.

10. The method of claim 1, further comprising use of the keratin layer for the in vitro culturing of cells.

11. The method as recited in claim 10, wherein the cells are animal or human epithelial or endothelial cells.

12. The method of claim 1, further comprising use of the keratin layer as recited in claim 1 for the manufacture of medical products for use as wound dressings.

13. The method of claim 1, further comprising use of the keratin layer as recited in claim 1 for the manufacture of an implant.

14. The method of claim 13, wherein the keratin layer is colonized at least in some segments with cells by the in vitro culturing.

15. The method of claim 14, wherein the keratin layer contains a softener.

16. The method as recited in claim 10, wherein the keratin layer contains a softener.

17. The method as recited in claim 11, wherein the keratin layer contains a softener.

18. The method as recited in claim 12, wherein the keratin layer contains a softener.

19. The method as recited in claim 13, wherein the keratin layer contains a softener.
